(19) United States
(12) Patent Application Publication
(10) Pub. No.: US 2006/0253028 A1
(43) Pub. Date: Nov. 9, 2006

(54) Multiple transducer configurations for medical ultrasound imaging

(75) Inventors: Duc H. Lam, San Jose, CA (US); Tat-Jin Teo, Sunnyvale, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: Scimed Life Systems, Inc.

(21) Appl. No.: 11/111,052

(22) Filed: Apr. 20, 2005

(51) Int. Cl. A61B 8/14

(52) U.S. Cl. 600/459

(57) ABSTRACT

The systems and methods described herein provide for multiple transducer configurations for use in medical ultrasound imaging systems. A medical device has a rotatable imaging device located therein for imaging an internal body lumen or cavity. The imaging device can include multiple transducers each configured to image a separate tissue depth or range of tissue depths. The transducers can be configured to operate over separate frequency ranges, with separate physical focuses or any combination thereof. Also provided is an image processing system configured to combine the image data collected from each transducer into a tissue image.

### FIELD OF THE INVENTION

The systems and methods relate generally to medical ultrasound imaging systems and, more particularly, to multiple transducer configurations for imaging wider depth ranges.

### BACKGROUND INFORMATION

Conventional medical ultrasound imaging systems, such as intravascular ultrasound (IVUS) and intracardiac echocardiography (ICE), use an ultrasound imaging device to image the interior of a living being. The ultrasound imaging device is placed on or within a catheter, which can then be inserted into the body for imaging a desired region, such as a body lumen, body cavity and the like. The ultrasound imaging device, which typically includes a transducer, is also communicatively coupled with an imaging system for processing and displaying any image data collected by the transducer. Ultrasound imaging systems can image with a number of different techniques, such as through the use of a rotatable transducer, a transducer array and the like.

In imaging systems that use a rotatable transducer, the transducer is typically mounted on the distal end of a rotatable driveshaft. The catheter typically includes an elongate tubular outer sheath configured to slidably receive the driveshaft. The driveshaft, along with the transducer mounted thereon, can then be rotated within the outer sheath. During rotation, the transducer transmits ultrasound signals into the surrounding lumen tissue. The tissue reflects these signals as echoes, which can then be received by the transducer.

The transducer then outputs an imaging signal indicative of the echo signal characteristics to the imaging system, which processes and stores the signal as an echogenic record. The transducer performs this imaging cycle, i.e., the process of transmitting an ultrasound signal or pulse and receiving the echoes generated therefrom, in a continuous manner as the transducer rotates. Multiple echogenic records are then accumulated by the imaging system, with each record typically corresponding to a different angular position of the transducer. The echogenic records can then be displayed as an image of the body lumen, such as a cross sectional image obtained during one rotation of the transducer. The transducer can be moved longitudinally within the outer sheath via the drive shaft, so that numerous locations along the length of the body lumen can be imaged.

Conventional transducers and other ultrasound imaging devices operate over a finite frequency bandwidth. The frequency of the ultrasound signal is a significant factor in determining the tissue depth that the transmitted ultrasound signal can penetrate. In general, lower frequency signals penetrate the tissue to a greater depth than higher frequency signals. Thus, a transducer operating in a lower frequency range is capable of producing an image at greater depths than a transducer operating at a higher frequency range.

However, the level of image quality produced at different depths is a complex interplay of numerous factors, such as overall system bandwidth (for example, the bandwidth of the receiving circuitry), transducer focus, beam pattern in addition to transducer frequency. All of these factors affect the axial and lateral size of the transmitted, or interrogating, pulse and change the size of the pulse as it propagates through the tissue. The pulse size can be considered one of the major factors affecting image quality. When designing a rotatable imaging device, the designer must select a transducer that can operate over a frequency range wide enough to allow imaging of the desired tissue depths, while at the same time balancing this against the other main performance affecting factors to arrive at a transducer design that produces a quality image.

Accordingly, improved ultrasound imaging systems are needed that can overcome the shortcomings of conventional imaging techniques while at the same time provide greater performance.

### SUMMARY

The systems and methods described herein provide for multiple transducer configurations for ultrasound imaging systems having an imaging device configured to image the interior of a living being. In one example embodiment of these systems and methods, the imaging device includes a first transducer and a second transducer, where the first transducer is configured to image a first range of depths and the second transducer is configured to image a second range of depths. Each transducer can be configured to image a range of depths by adjusting the transducer's physical focus or by adjusting the transducer's operating frequency or any combination thereof.

The imaging system can also include an image processing system communicatively coupled with the transducer devices and configured to receive a first output signal from the first transducer and a second output signal from the second transducer. The image processing system can be configured to process the first and second output signals into image data and combine the image data such that the image data is displayable as a single image.

In another example embodiment of the systems and methods described herein, the first transducer is configured to operate over a first frequency range and output a first output signal to the image processing system over a signal line. The second transducer is configured to operate over a second frequency range and output a second output signal to the image processing system over the same signal line. The image processing system can be configured to separate the first and second output signals, for instance, by using a signal separation unit and the like.

In another example embodiment of the systems and methods described herein, the first transducer is positioned in the imaging device at a first location and the second transducer is positioned in the imaging device at a second location opposite the first location. The location of the first and second transducers within the imaging device is preferably symmetrical.

In yet another embodiment of the systems and methods described herein, an image processing system is configured to receive a first transducer output signal and process the first output signal into a first echogenic data set comprising a plurality of image data items collected over a first range of tissue depths. The image processing system is also configured to receive a second transducer output signal and process the second output signal into a second echogenic data set comprising a plurality of image data items collected over a second range of tissue depths. The image processing system is further configured to combine the first and second echogenic data sets such that the image data items in the first and second ranges of tissue depths are displayable as a single image.

The first echogenic data set and the second echogenic data set may each comprise at least one data item collected from the same tissue depth. The image processing system can be configured to blend each data item from the first echogenic data set with each data item from the second echogenic data set collected at the same tissue depth to produce a blended data item.

In still another embodiment, the image processing system can be configured to receive a first transducer output signal over a first time period and a second transducer output signal over a second time period. The image processing system can also be configured to ignore the second output signal during the first time period.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims. It is also intended that the invention is not limited to the details of the example embodiments.

### BRIEF DESCRIPTION OF THE FIGURES

The details of the invention, both as to its structure and operation, may be gleaned in part by study of the accompanying figures, in which like reference numerals refer to like parts. The components in the figures are not necessarily to scale, emphasis instead being placed upon illustrating the principles of the invention. Moreover, all illustrations are intended to convey concepts, where relative sizes, shapes and other detailed attributes may be illustrated schematically rather than literally or precisely.

FIG. 1 is a perspective view depicting an example embodiment of an ultrasound imaging system.

FIG. 2 is a graph depicting example operating frequency ranges for two transducers within an example embodiment of the ultrasound imaging system.

FIGS. 3A-B are schematic views depicting example embodiments of an ultrasound imaging device.

FIGS. 4A-B are timing diagrams depicting the operation of one example embodiment of the ultrasound imaging system having two transducers.

FIG. 5A is an example intravascular ultrasound image.

FIG. 5B is an illustration depicting an example embodiment of data collected in a portion of the example image depicted in FIG. 5A.

FIG. 6A is a perspective view depicting another example embodiment of an ultrasound imaging system.

FIG. 6B is a block diagram depicting another example embodiment of an ultrasound imaging system.

### DETAILED DESCRIPTION

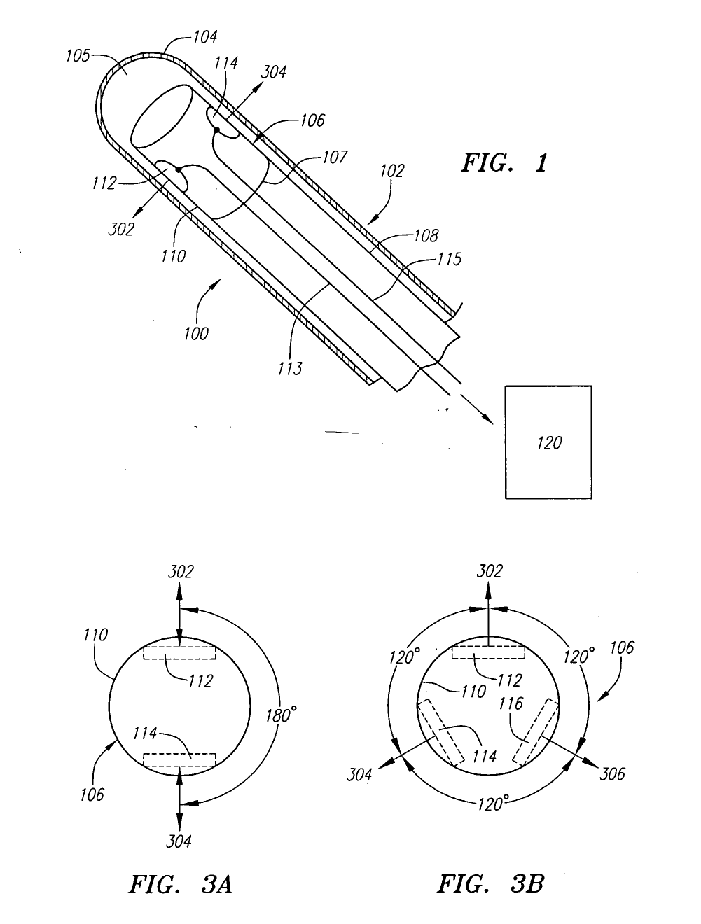

The systems and methods described herein provide for multiple transducer configurations in ultrasound imaging systems. These systems and methods allow an ultrasound imaging system to image a greater range of tissue depths while maintaining a relatively high degree of image quality. FIG. 1 depicts a schematic diagram of one example embodiment of an ultrasound imaging system 100 for use with the systems and methods described herein. Preferably, imaging system 100 is an IVUS imaging system, although the systems and methods are not limited to such and any other type of imaging system, such as ICE, can be used. Here, catheter 102 is shown having elongate tubular outer sheath 104 and inner lumen 105. An imaging device 106 is preferably mounted on distal end 107 of rotatable driveshaft 108, which is configured to move, or slide, within inner lumen 105. System 100 is preferably configured to image a tissue cross-section by rotating imaging device 106, although system 100 is not limited to rotational techniques. Imaging device 106 preferably includes housing 110 and two transducers 112 and 114.

Transducers 112 and 114 are preferably configured to image different tissue depths, or ranges of tissue depths. Transducers 112 and 114 are preferably communicatively coupled with image processing system 120 via communication paths 113 and 115, respectively. During an imaging procedure, each transducer 112 and 114 can be operated to obtain separate image data sets containing image data from different tissue depths. Imaging system 120 can be configured to compile and process these image data sets such that they are displayable as a single high quality image covering a wider tissue depth range than conventional systems.

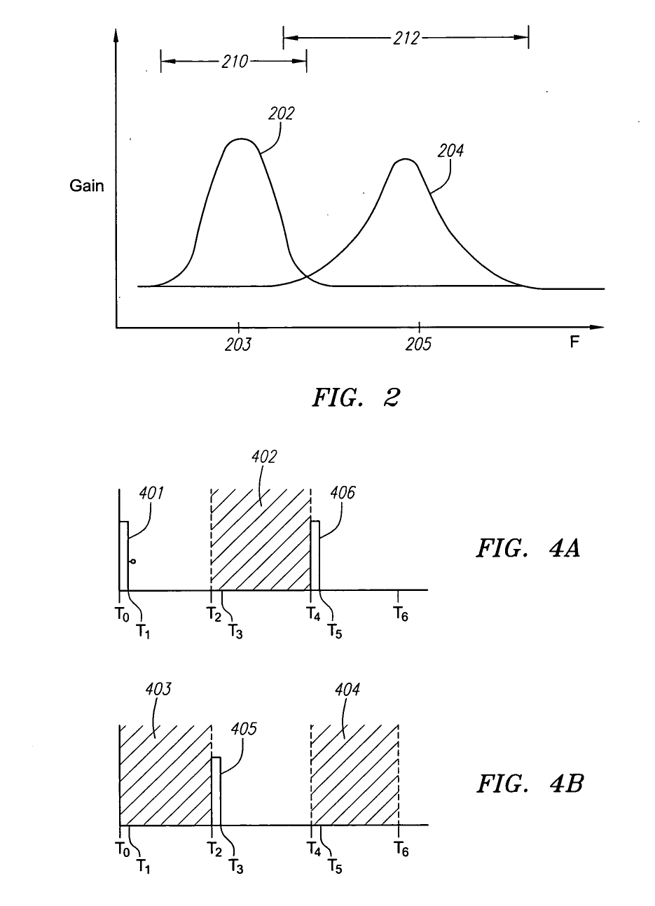

Using various methods, transducer 112 and 114 can be configured to image different tissue depths, which can be either overlapping or non-overlapping. For instance, transducers 112 and 114 can be configured to operate over different frequency ranges, or with different physical focuses, or with any combination of the two. In one embodiment, transducers 112 and 114 are configured to operate over different bandwidths, or frequency ranges. Here, for example, each transducer 112 and 114 is preferably configured to operate at a separate center frequency with partially overlapping bandwidths as depicted in FIG. 2.

FIG. 2 depicts example frequency response 202 for transducer 112 having bandwidth 210 and center frequency 203 along with example frequency response 204 for transducer 114 having bandwidth 212 and center frequency 205. The amount of bandwidth overlap can be varied according to the needs of the application. By adjusting the other design aspects, such as focus, beam pattern and the like, each transducer 112 and 114 can be optimized to image the respective range of tissue depths. Center frequencies 203 and 205 and bandwidths 210 and 212 can be chosen based on the needs of the application. For example, in one embodiment, center frequencies 203 and 205 are 40 Megahertz (Mhz) and 80 Mhz respectively, while bandwidths 210 and 212 are 18 Mhz-62 Mhz and 58 Mhz-102 Mhz, respectively. It should be noted that these values are used only as an example and in no way limit the systems and methods described herein.

Alternatively, imaging system 100 can be configured such that transducers 112 and 114 each have a different physical focus to image a different range of tissue depths. Physical focus can be adjusted by changing the shape of the transducer, adding a lens to the transducer and the like. Preferably, the depth ranges for each transducer 112 and 114 at least partially overlap, although this is not required. The tissue depth focus chosen for each transducer 112 and 114 will depend on the needs of the application. For instance, in intracardiac applications, the distance from the imaging device 106 to the body lumen or heart chamber is typically on the order of one to two centimeters, while in coronary applications, the distance from the imaging device to the body lumen is typically 4 millimeters or less.

FIG. 3A depicts a schematic top down view of an example embodiment of imaging. device 106 with transducers 112 and 114 housed therein. In this embodiment, transducers 112 and 114 are positioned radially around a center axis of catheter 102 . Transducers 112 and 114 have an angular separation of 180 degrees such that transducers 112 and 114 are aligned in opposite directions. Arrows 302 and 304 indicate the primary directions in which transducers 112 and 114, respectively, transmit and receive ultrasound energy. One of skill in the art will readily recognize that each transducer 112 and 114 also transmits and receives ultrasound energy in directions adjacent to or close to these primary directions 302 and 304, although energy transmitted and received in these other directions can be greatly reduced.

During operation of system 100, each transducer 112 and 114 transmits and receives ultrasound energy in these primary directions 302 and 304, respectively. When operating within a body lumen, each transducer 112 and 114 effectively images regions of the lumen located opposite to each other. Because each transducer 112 and 114 is preferably configured to image a different range of depths, as imaging device 106 performs a rotation, image data from each transducer 112 and 114 is obtained and can be combined by IVUS imaging system 100 to produce a single cross-sectional image of the body lumen showing a wider range of depths.

Although the above described embodiments of imaging system 100 have two transducers 112 and 114, any number of transducers can be used. For instance, FIG. 3B depicts an example embodiment of imaging device 106 having three transducers 112, 114 and 116, where each transducer 112-116 is configured to operate over a different range of frequencies. For embodiments where imaging device 106 is rotated during the imaging procedure, the transducers 112-116 are preferably placed in a symmetrical arrangement within housing 107. Here, each transducer 112-116 is placed 120 degrees apart to form the symmetrical arrangement, whereas in the embodiment depicted in FIG. 3A, transducers 112 and 114 are placed 180 degrees apart to form the symmetrical arrangement. The symmetrical arrangement is advantageous for purposes of minimizing non-uniform rotational distortion (NURD), which may be more likely to occur in asymmetric arrangements. One of skill in the art will readily recognize that the arrangement does not require absolute symmetry and substantially symmetric arrangements can be used. Here, substantial symmetry refers to any arrangement that reduces the risk of NURD to a level acceptable for the needs of the application.

The embodiment in FIG. 3A is preferred because the opposite alignment of transducers 112 and 114 minimizes the potential for cross-talk during the operation of each transducer 112 and 114. In the embodiment depicted in FIG. 3B, the potential for cross-talk between transducers 112-116 is increased, since the primary operating directions 302-306 are not directly opposite as in the embodiment depicted in FIG. 3A. Furthermore, the potential for cross-talk would be even greater in an embodiment having four transducers placed with 90 degrees of separation between them. Thus, the amount of allowable cross-talk in the application should be taken into account when designing imaging device 106. One of skill in the art will readily recognize that the effects of cross-talk can be minimized through the use of filtering circuitry and the like within image processing system 120.

It should be understood that the needs of each application will vary, and that the systems and methods described herein are not limited to any one configuration of transducers. For instance, a dual transducer “bullseye” configuration having an inner transducer surrounded by an outer, annular transducer is just one example of another configuration that can be implemented in system 100.

Furthermore, the IVUS imaging system 100 can be configured such that each transducer is operative, i.e., transmitting or receiving, in separate time segments. FIGS. 4A-B depict timing diagrams for an example embodiment of IVUS imaging system 100 having two transducers 112 and 114, which preferably rotate continuously during the imaging procedure. FIG. 4A depicts a timing diagram for transducer 112, while FIG. 4B depicts a timing diagram for transducer 114. In FIG. 4A, at time T₀, transducer 112 transmits an ultrasound pulse 401. From time T₁ to T₂, transducer 112 receives ultrasound echoes generated from the transmission of pulse 401. During time period 403 from time T₀ to T₂, transducer 114 is non-operative. i.e., neither transmitting or receiving for the purpose of collecting data, and image processing system 120 is configured to ignore any echoes received from transducer 114 during this time 403. At time T₂, transducer 114 becomes operative and transmits ultrasound pulse 405 and listens for resulting echoes from time T₃ to T₄. During time period 402 from T₂ to T₄, transducer 112 is non-operative and image processing system 120 is configured to ignore any echoes received during this time 402.

Image processing system 120 can be configured to ignore signals received by the non-operative transducer 112 or 114 in any manner, including the use of hardware or software implementations. At time T₄, imaging device 106 has rotated to a new angular position so that the imaging process can be repeated. One of skill in the art will readily recognize that other embodiments can be configured with more than two transducers 112 and 114 by adding an additional time period for each additional transducer where that transducer is operative and the image processing system 120 ignores echoes received by the other transducers.

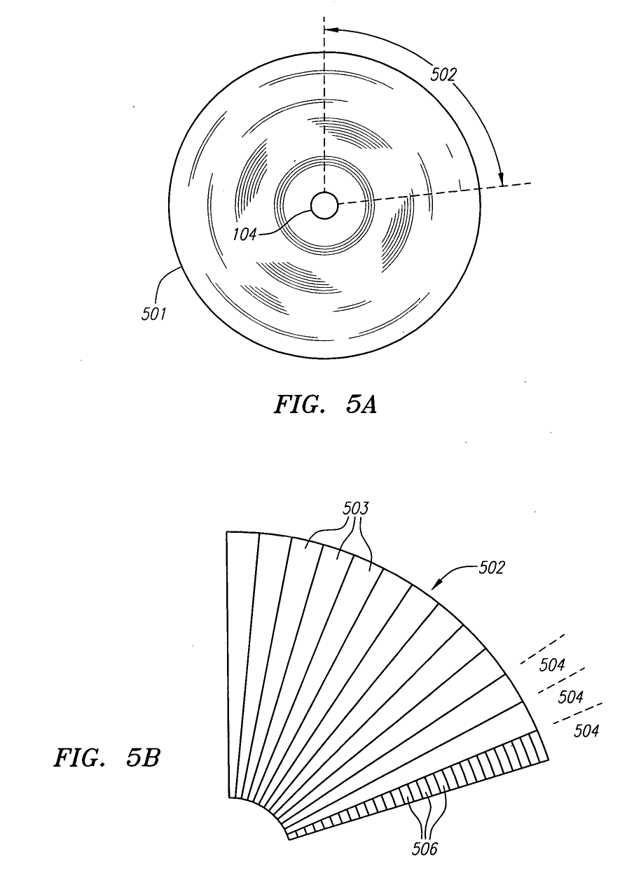

FIG. 5A depicts an example ultrasound image 501 of a body lumen. FIG. 5B depicts a block diagram of section 502 of image 501 showing example data collecting by imaging system 100 for the body lumen. Here, multiple individual echogenic records 503 are depicted, each located at a separate angular position 504. Each echogenic data record 503 includes data representative of the echoes received by one transducer in response to an ultrasound pulse transmitted at that angular position 504. In an embodiment having two transducers 112 and 114, imaging system 100 preferably stores one echogenic data record 503 for each angular position 504 of each transducer 112 and 114 and each transducer 112 and 114 preferably images the same or similar angular positions 504. In one example embodiment, IVUS imaging system 100 collects 360 echogenic data records 503 during one rotation, with one echogenic data record 503 for every degree of rotation.

Within each echogenic data record 503 are individual data items 506. Each data item 506 has data representative of the strength of an echo received from a certain depth. This data can be used, for instance, to determine a brightness value for the image. Various tissue features reflect the incident ultrasound pulse differently and will translate into echoes of various strengths. In one embodiment, the depth of the tissue feature is determined, for instance, by the time delay between the transmission of the ultrasound pulse and receipt of the echo. The tissue depth and angular position 504 correlate to a position on image 501. The strength of the received echo can be translated into a brightness value for that position on image 501. In this manner, image 501 of the body tissue can be constructed.

In one embodiment, echogenic data sets 503 for each transducer 112 and 114 are compiled into an image data set. Echogenic data records 503 from corresponding angular positions in each image data set are then combined, or blended, to form a combined image data set. Data items 506 occurring at similar depths and angular positions 504 are combined, or blended, in a manner sufficient to produce a resulting blended data item. A simple additive combination of data items 506 would not accurately reflect the corresponding tissue feature because, for instance, the resulting data item 506 would be an additive combination of two signals received from the same tissue feature.

The blended data item preferably accurately represents the tissue feature in relation to the other tissue features in image 501. Any method process, or technique of combining or blending ultrasound data can be used. For instance, in one embodiment, data items 506 occurring at the same depth and angular position 504 are averaged. Another method of data blending is disclosed in U.S. Pat. No. 6,132,374 issued to Hossack et al. on Oct. 17, 2000, which is fully incorporated by reference herein. By combining the ultrasound data, an ultrasound image 501 showing tissue features occurring over a wide range of depths can be generated. Imaging system 100 can combine the image data as each data item 506 is collected, as each echogenic data record 503 is collected or after any number of echogenic data records 503 are collected as needed by the application.

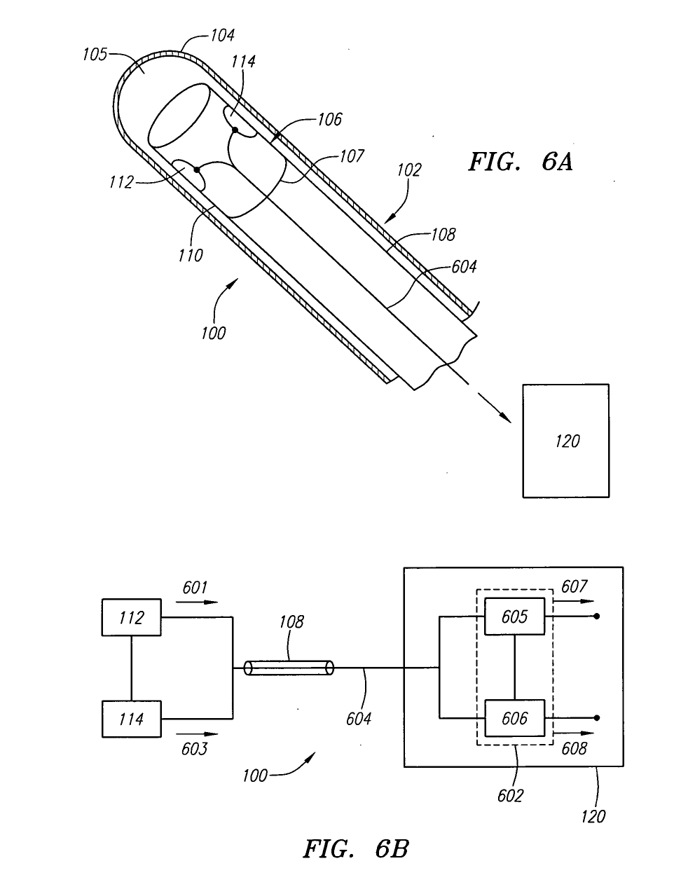

FIG. 6A depicts a schematic diagram of another example embodiment of IVUS imaging system 100 where transducers 112 and 114 are configured to operate over different frequency ranges. Here, transducers 112 and 114 share a common communicative path 602 with image processing system 120. Each transducer 112 and 114 outputs an imaging signal at frequencies within that transducer's frequency range of operation. Preferably, the frequency ranges for each transducer 112 and 114 are sufficiently separate to allow image processing system 120 to receive each output signal independently. In this embodiment, image processing system 120 includes a signal separation unit 602 for separating the output signals received from each transducer 112 and 114.

FIG. 6B is a block diagram depicting one example embodiment of signal separation unit 602 using bandpass filter circuitry. In this embodiment, output signals 601 and 603 from transducers 112 and 114, respectively, travel along communicative path 604 to bandpass filters 605 and 606. Bandpass filter 605 is configured to filter all signals having frequencies except those within the frequency range of transducer 112, while bandpass filter 606 is configured to filter all signals having frequencies except those within the frequency range of transducer 114. Signals 607 and 608 output from each filter 605 and 606, respectively, can then be interpreted by image processing system 120 as being representative of output signals 601 and 603.

By using signal separation unit 602, transducers 112 and 114 can share a common communicative path, which can allow the size of drive shaft 108 and outer sheath 104 to be reduced. As a result, catheter 102 can be advanced into smaller body lumens. One of skill in the art will readily recognize that signal separation can be implemented in numerous ways and with numerous circuitry types other than bandpass filters. For instance, a highpass and lowpass filter combination can be used, as well as certain algorithmic and software techniques and the like.

In the foregoing specification, the invention has been described with reference to specific embodiments thereof. It will, however, be evident that various modifications and changes may be made thereto without departing from the broader spirit and scope of the invention. For example, each feature of one embodiment can be mixed and matched with other features shown in other embodiments. Features and processes known to those of ordinary skill may similarly be incorporated as desired. Additionally and obviously, features may be added or subtracted as desired. Accordingly, the invention is not to be restricted except in light of the attached claims and their equivalents.

What is claimed is:

1. A medical ultrasound imaging system, comprising:
an imaging device configured to image an internal body lumen, the imaging device comprising:
a first transducer; and
a second transducer, wherein the first transducer is configured to image a first range of depths and the second transducer is configured to image a second range of depths.

2. The system of claim 1, wherein the first transducer is focused to image the first range of depths and the second transducer is focused to image the second range of depths.

3. The system of claim 2, further comprising:
a rotatable driveshaft having the imaging device coupled thereto; and
an elongate tubular member having an inner lumen configured to slidably receive the rotatable driveshaft.

4. The system of claim 3, wherein a signal line is coupled with the first transducer and the second transducer, and the first transducer is configured to output a first output signal over the signal line and the second transducer is configured to output a second output signal over the signal line.

5. The system of claim 4, further comprising an image processing system communicatively coupled with the first and second transducers, wherein the image processing system is configured to alternately receive the first and second output signals, process the first and second output signals into image data and combine the image data from the first and second output signals for display as a single image.

6. The system of claim 1, wherein the first transducer is configured to operate over a first frequency range and the second transducer is configured to operate over a second frequency range.

7. The system of claim 6, wherein the first and second frequency ranges do not overlap.

8. The system of claim 7, further comprising:
a rotatable driveshaft having the imaging device coupled thereto; and
an elongate tubular member having an inner lumen configured to slidably receive the rotatable driveshaft, wherein a signal line is coupled with the first transducer and the second transducer, and wherein the first transducer is configured to output a first output signal over the signal line and the second transducer is configured to output a second output signal over the signal line.

9. The system of claim 8, further comprising an image processing system communicatively coupled with the first and second transducers over the signal line, wherein the image processing system is configured to process the first and second output signals.

10. The system of claim 6, wherein the image processing system comprises a signal separation unit configured to separate the first and second output signals.

11. The system of claim 6, wherein the first and second frequency ranges partially overlap.

12. The system of claim 11, further comprising:
a rotatable driveshaft having the imaging device coupled thereto; and
an elongate tubular member having an inner lumen configured to slidably receive the rotatable driveshaft.

13. The system of claim 12, wherein the first transducer is configured to output a first output signal over a first signal line coupled thereto, and wherein the second transducer is configured to output a second output signal over a second signal line coupled thereto.

14. The system of claim 12, wherein a signal line is coupled with the first transducer and the second transducer, and wherein the first transducer is configured to output a first output signal over the signal line and the second transducer is configured to output a second output signal over the signal line.

15. The system of claim 14, further comprising an image processing system communicatively coupled with the first and second transducers over the signal line, wherein the image processing system is configured to process the first and second output signals.

16. The system of claim 15, wherein the image processing system comprises a signal separation unit configured to separate the first and second output signals.

17. The system of claim 12, further comprising an image processing system communicatively coupled with the first and second transducers, wherein the first transducer is configured to output a first output signal to the image processing system and the second transducer is configured to output a second output signal to the image processing system and wherein the image processing system is configured to process the first and second output signals into image data and combine the image data from the first and second output signals for display as a single image.

18. The system of claim 1, wherein the first transducer is positioned in the imaging device at a first location and the second transducer is positioned in the imaging device at a second location opposite the first location.

19. The system of claim 1, wherein the first and second transducers are configured to image in opposite directions.

20. The system of claim 19, wherein the first and second transducer are positioned substantially symmetrically.

21. The system of claim 1, further comprising a third transducer, wherein the first transducer, second transducer and third transducer are substantially symmetrically positioned in the imaging device.

22. The system of claim 21, wherein the first transducer is configured to operate over a first frequency range, the second transducer is configured to operate over a second frequency range and the third transducer is configured to operate over a third frequency range.

23. The system of claim 22, wherein each transducer is communicatively coupled with an image processing system over a common signal line.

24. A medical ultrasound imaging system, comprising:
an image processing system configured to receive a first transducer output signal and process the first output signal into a first echogenic data set comprising a plurality of image data items collected over a first range of tissue depths, and configured to receive a second transducer output signal and process the second output signal into a second echogenic data set comprising a plurality of image data items collected over a second range of tissue depths, wherein the image processing system is further configured to combine the first and second echogenic data sets for display as a single image.

25. The system of claim 24, wherein the first echogenic data set and the second echogenic data set each comprise at least one data item collected from the same tissue depth.

26. The system of claim 25, wherein the image processing system is configured to blend each data item from the first echogenic data set with the data item from the second echogenic data set collected at the same tissue depth to produce a blended data item.

27. The system of claim 26, wherein the first output signal is received over a first frequency range and the second output signal is received over a second frequency range.

28. The system of claim 27, wherein the first frequency range and the second frequency range do not overlap.

29. The system of claim 27, wherein the first frequency range and the second frequency range at least partially overlap.

30. The system of claim 27, wherein the image processing system is configured to separate the first output signal from the second output signal.

31. The system of claim 27, further comprising a signal separation unit configured to separate the first output signal from the second output signal.

32. The system of claim 26, wherein the image processing system is configured to receive the first output signal over a first time period and the second output signal over a second time period.

33. The system of claim 32, wherein the image processing system is configured to ignore the second output signal during the first time period.

34. A method of ultrasound imaging, comprising:
receiving a first output signal from a first ultrasound transducer located within a living being, the first output signal being representative of a first echo received by the first transducer from a first range of depths in the living being; and
receiving a second output signal from a second ultrasound transducer located within the living being, the second output signal being representative of a second echo received by the second transducer from a second range of depths in the living being, wherein the first and second range of depths are at least partially different.

35. The method of claim 34, wherein the first and second output signals are at substantially the same frequency.

36. The method of claim 35, further comprising:
storing the first output signal and the second output signal in a first echogenic record and a second echogenic record, respectively; and
processing the first and second echogenic records into an image of the living being, the image covering the first and second ranges of depths, wherein the first and second output signals are received alternately over a common signal line.

37. The method of claim 35, further comprising:
storing the first output signal and the second output signal in a first echogenic record and a second echogenic record, respectively; and
processing the first and second echogenic records into an image of the living being, the image covering the first and second ranges of depths, wherein the first and second output signals are received over a first and a second signal line, respectively.

38. The method of claim 34, wherein the first output signal is at a first frequency range and the second output signal is at a second frequency range at least partially overlapping the first frequency range.

39. The method of claim 38, further comprising:
storing the first output signal and the second output signal in a first echogenic record and a second echogenic record, respectively; and
processing the first and second echogenic records into an image of the living being, the image covering the first and second ranges of depths, wherein the first and second output signals are received over a common signal line.

40. The method of claim 38, further comprising:
storing the first output signal and the second output signal in a first echogenic record and a second echogenic record, respectively; and
processing the first and second echogenic records into an image of the living being, the image covering the first and second ranges of depths, wherein the first and second output signals are received over a first and a second signal line, respectively.

41. The method of claim 34, wherein the first output signal is at a first frequency range and the second output signal is at a second frequency range different from the first frequency range.

42. The method of claim 41, further comprising:
storing the first output signal and the second output signal in a first echogenic record and a second echogenic record, respectively; and
processing the first and second echogenic records into an image of the living being, the image covering the first and second ranges of depths, wherein the first and second output signals are received over a common signal line.

43. The method of claim 41, further comprising:
storing the first output signal and the second output signal in a first echogenic record and a second echogenic record, respectively; and
processing the first and second echogenic records into an image of the living being, the image covering the first and second ranges of depths, wherein the first and second output signals are received over a first and a second signal line, respectively.
